(19) United States
(12) Patent Application Publication
(10) Pub. No.: US 2019/0313722 A1
(43) Pub. Date: Oct. 17, 2019

(54) POSTURE GARMENT WITH TWO-WAY STRETCH UTILIZING BILATERAL, VARIABLE TENSION TO ALIGN THORACIC and LUMBAR SPINE

(71) Applicant: IFGCURE HOLDINGS, LLC, LOS ANGELES, CA (US)

(72) Inventor: Stephen H. LIU, LOS ANGELES, CA (US)

(21) Appl. No.: 16/290,845

(22) Filed: Mar. 1, 2019

Related U.S. Application Data
(60) Provisional application No. 62/637,138, filed on Mar. 1, 2018.

(51) Int. Cl. A41F 9/02; A41D 1/04; A61F 5/02

(52) U.S. Cl. CPC A41F 9/025; A41D 1/04; A41D 2400/38; A41D 2300/32; A61F 5/028

(57) ABSTRACT

A manufactured garment in the posture correction and athletic enhancement space. A garment that is uniquely constructed with modern performance of fabrics of a breathable, spandex poly blend, and includes numerous internal bands connected to a mechanical tensioning system, which reduces weight and creates intracavitary pressure, thus leading to individualized spinal alignment that is customizable for each user.

### BACKGROUND OF THE INVENTION

### Field of the Invention

Embodiments of the present invention are directed to a garment in the posture correction and athletic enhancement space. It is a garment that is uniquely designed, manufactured, and constructed with modern performance fabrics of a breathable, spandex poly blend, and includes numerous internal bands connected to a mechanical tensioning system, which reduces weight and creates intracavitary pressure, thus leading to individualized spinal alignment that can be customizable per patient.

### Description of the Related Art

Posture correcting shirts are in existence and been used for several years, the primary goal being to stimulate body muscles into better alignment and posture (a slight S-shape of the spine being the gold standard). The importance of having good posture is a well-known and accepted idea among health professionals and non-experts in the field. Good posture helps for both health reasons (back, shoulder, neck pain, etc.) and athletic performance, as poor posture during dynamic activities results in inefficient biomechanics and body movement. Inefficient body movement does not allow for full utilization of muscular range of motion and strength and can cause repetitive injury over time.

Injuries to a person's body muscles can often be debilitating and can prevent workers from going about their everyday jobs. Certain injuries can also lead to a permanently hunched posture. Typical recovery methods for those suffering from back, neck, and other pain (including injuries) include going to a chiropractor or physical therapy or to seek orthopedic surgeon evaluation. Such medical procedures to correct poor posture involve injections, medications, rehabilitation, and possible surgical correction. As many people cannot afford the time or cost of extensive and costly chiropractic treatments (either short term or long-term treatments), there exists a need in the market for affordable methods and systems to correct and maintain the posture of individuals in attempt to provide:

a) correct functional anatomy;

b) improved muscle efficiency;

c) improved pain relief and minimal pain to users; and

d) the creation of good habits so that individuals do not develop poor-posture related pain to begin with.

As one treatment option, posture shirts were created to fill this need of proprioceptive therapy. Posture shirts typically contain elastic bands and special stitching that helps maintain an upright body with correct alignment both skeletal and muscle-wise through the contraction of certain muscles. In 2006, Alignmed created a shirt made from a cotton body with elastic bands that were attached at the front of each shoulder, ran over the back parallel to the spine, and connected at the bottom seam. The Alignmed shirt is made from a Lycra Spandex material with mesh and a woven stretch fabric representing the original bands in that first version posture shirt that Alignmed created in 2006. Competitor companies also use this same Lycra Spandex combination material to provide structural stability, compression, and an athletic garment look; however, this fabric is not breathable. In order to be effective at gaining stability from the body for proprioception and to correct posture imbalance, Lycra Spandex made garments must be worn extremely tight, leading to discomfort.

Not only does Lycra Spandex material require an extremely tight wear from the user, it also must contain mesh to provide for ventilation and breathability. This mesh contributes to creating an unsightly and unfashionable look that does not translate well to commercial use, since customers are not inclined to wear the garment as their only layer. By wearing an additional layer on top of the Lycra Spandex to cover the unsightly mesh posture correction garment, the breathability issue is compounded with an additional layer of tightness. Due to this combination, users typically stop wearing Lycra Spandex made posture shirts, thus defeating the purpose of building good habits in posture and natural, straight alignment.

To correct for some of these flaws, competitors created what is known in the market as the Spinal-Q body jacket. This heavy-laden jacket utilizes friction bands that extend from the pectoral areas at opposite sides, where they are anchored to the interior of a garment, to extend over the respective shoulders, then attaching to tension bands which pass diagonally across the back (in a figure 8 shape) and exit the opposite sides of the garment to attach at the front of the pelvis. While this has been effective in correcting for spinal alignment, the Spinal-Q body jacket still has its drawbacks, namely: it can only correct for mid-thoracic misalignment in an anterior/posterior plane of motion; it is made from a 360-degree knitted poly that creates a tube-sock like method of construction, thus limiting mobility and becoming more highly visible; and it contains only one selection of spinal adjustment.

Taking these facts into consideration, there exists a need for a more comfortable and fashionable garment that utilizes a more breathable fabric, allows for greater mobility, and can offer varying degrees of spinal alignment to correct for multiple patient needs.

Accordingly, embodiments of the present invention are directed to solving these problems.

### SUMMARY OF THE INVENTION

Objects of the invention are achieved by providing a manufactured garment that accounts for and overcomes the limitations of the currently made Lycra Spandex and Spinal-Q garments.

Objects of the invention are achieved by providing a comfortable and fashionable garment that utilizes a more breathable fabric, allows for greater mobility, and can offer varying degrees of spinal alignment to correct for multiple patient needs.

These and other objects of the invention are achieved by providing a breathable, modern, muscle rebalance performance garment, comprising: a garment body made of a breathable spandex poly blend; at least one internal stretch band arranged horizontally across the garment body; and a mechanical tensioning system comprised of a spool, a cable, a channel, a cam, and a tension wheel, wherein the mechanical tensioning system is used to tension the at least one internal stretch band to customize the tension of the garment.

In certain embodiments, the present invention is directed to a garment, comprising: a plurality of garment regions; a mechanical tensioning system comprising a spool and a cable; and at least one band comprising a first and a second end, said first end attached along a longitudinal center line of said garment and said second end attached to said mechanical tensioning system, wherein said plurality of regions are individually adjustable using said mechanical tensioning system to provide a customized tension fit.

In certain embodiments, the at least one band is stacked from the lumbar spine to the thoracic.

In certain embodiments, the at least one band extends through at least one internal loop, so that the at least one band is secured to the garment body yet is still able to move and stretch independently.

In certain embodiments, one end of the band is securely attached via a seam to the front of garment center line and the other end is attached to the mechanical tensioning system.

In certain embodiments, the at least one internal stretch band creates intracavitary pressure and thus spinal alignment in a wearer of the garment.

In certain embodiments, when the spool is tightened, the cable shortens and applies torque to the at least one band.

In certain embodiments, not only can each band be individually adjusted to apply the desired amount of pressure, but also, each side of the garment can also be independently adjusted to create different amounts of tension in the garment.

In certain embodiments, each side of the garment is able to have its tension controlled by the mechanical tensioning system.

In certain embodiments, the mechanical tensioning system of bands provides support to the wearer.

In certain embodiments, the mechanical tensioning system of bands provides vertical support to the wearer to support the wearer's posture.

In certain embodiments, the mechanical tensioning system of bands is customizable per wearer and adjustable for both tension and band length.

In certain embodiments, correction of a wearer's posture is corrected using bilateral, intracavitary pressure.

In certain embodiments, the garment corrects a wearer's posture.

In certain embodiments, the garment improves athletic performance.

In certain embodiments, the garment aligns the entire thoracic and lumbar spine.

In certain embodiments, the garment contains anti-microbial moisture wicking and is protects against ultra-violet (UV) rays.

In certain embodiments, the garment allows for greater range of motion and flexibility.

In certain embodiments, the garment is 31% lighter than the competitor in the market, the Spinal-Q.

In certain embodiments, the garment is highly variable and adjustable.

In certain embodiments, the garment is body temperature regulating.

In certain embodiments, the garment is bug proof and rapid-drying.

In certain embodiments, the garment may be a vest, a waistband, or a girdle.

In certain embodiments, the garment improves the ability to breathe for the wearer.

In certain embodiments, the garment improves lower back pain in the wearer.

In certain embodiments, the present invention is directed to a tensioning system, comprising: a tension wheel; a main cable wound around said tension wheel; a plurality of sub-cables attached to said main cable; at least two bands each attached to at least one of said plurality of sub-cables; and a cam positioned between said at least two bands comprising a plurality of holes, said main cable extending through a first and second of said holes and said sub-cables extending through a third and fourth of said holes, wherein said at least two bands are adjustable by turning said tension wheel.

In certain embodiments, the present invention is directed to a method for using the garment, comprising: adjusting a plurality of regions of said garment by adjusting a length of at least one band within at least one of said regions; securing said garment to the body of an individual; and adjusting the tension applied to at least one of said plurality of regions by a mechanical tensioning system to provide a customized tension fit.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is an anterior view of an embodiment of the garment of the present invention.

FIG. 2 is the posterior view of the garment of FIG. 1.

FIG. 3 is a close-up view of band system of the garment of FIG. 1.

FIG. 4 is a close-up view of the mechanical tensioning system of the garment of FIG. 1.

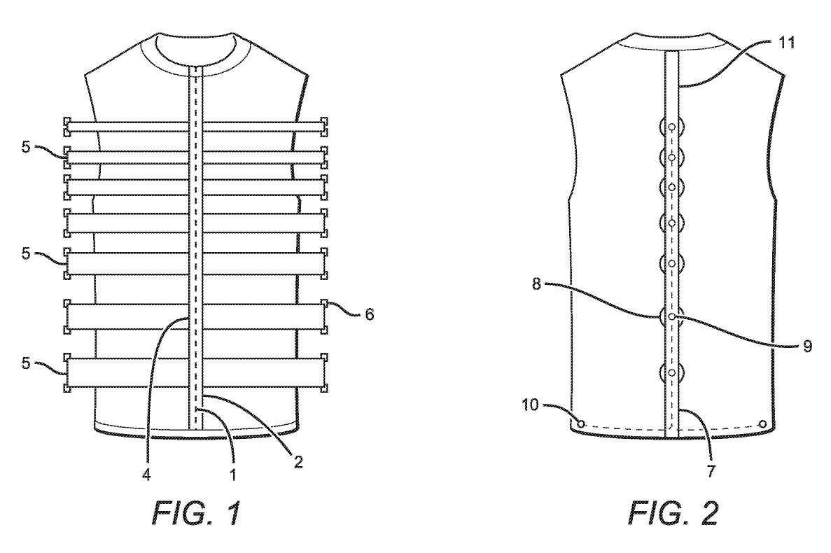

### DETAILED DESCRIPTION OF THE INVENTION

One embodiment of the present invention is a manufactured garment that accounts for and overcomes the limitations of currently available Lycra Spandex and Spinal-Q garments. The garment utilizes modern performance fabrics and a mechanical tensioning system to create intracavitary pressure in order to improve spinal alignment and muscular rebalance as well as increase athletic performance. The garment also utilizes bi-lateral, variable tension, which aligns the entire thoracic and lumbar spine and allows for more specific alignment possibilities through adjustable, individual bands.

In one embodiment, the garment may be created using a suitable blend of fabric such as, for example, 82% poly, 10% spandex, 8% tensile. Some suitable blends provide a garment that is moisture wicking, antimicrobial, protective against UV rays, bug proof, rapid drying, and unlike competitors such as the Spinal Q, breathable. The fabric also provides four-way stretch; in other words, the further the fabric is stretched, the greater its compression qualities. The garment also has a lower profile and can be worn under a shirt and not be seen, leading to greater mobility with body temperature regulation as opposed to the insulating fabric of the Spinal Q. In addition, it is also 31% lighter than the Spinal Q. It is understood, however, that many different materials and material combinations may be used to create the garment in order to achieve a particular set of characteristics.

One advantage of the garment is a product of the bilateral, variable tension that is created by allowing each side of the garment to be adjusted independently. For instance, if a patient suffers from a right side deviation of the thoracic spine that is a certain X degrees and a left side deviation of the lumbar that is a different certain Y degrees, the garment can be adjusted using internal bands that account for and pulls both deviations back to the center, creating spinal alignment that can be customized on a per patient basis. This customizable spinal alignment cannot not be achieved without the specially designed, vertically spaced bands that create the intracavitary pressure and thus spinal alignment.

The bands are arranged horizontally across each side of the garment. They are spaced approximately 1.5 inches apart and are stacked from the lumbar spine to the thoracic. Each band is fed through internal loops so that they are secured to the body of the garment yet still able to move and stretch independently. One end of the band is securely attached via a seam to the front of garment center line. The other end is attached to a tension system that consists of a self-locking spool and thin cable system. The cable is fed through a channel sewn into the garment. As the spool is tightened, the cable shortens and applies torque to the band. This is the tightening mechanism. Not only can each band be individually adjusted to apply the desired amount of pressure, but also, each side of the garment can also be independently adjusted to create different amounts of tension.

This spool and cable, mechanical tensioning system controls the overall gross tension of each side of the garment. A wearer can turn the spools upon wearing the garment to achieve a desired level of tension across bands sewn into the garment. Also, a wearer can achieve greater individual adjustment by adjusting the length of the bands prior to donning the garment. Each band has micro adjustment tabs that can be constructed from a hook-and-loop fastening mechanism (e.g., Velcro brand fastening mechanism) or can be designed with micro clips—small clips adhered to the end and sides of the band tail. The embodiments described herein reference the micro clip fastening mechanism, but it is understood that the fastening mechanism can comprise other fastening mechanisms such as the previously mentioned hook-and-loop fastening mechanism, or other fastening mechanisms. The band is fed through the cable system and secured back to itself with the micro clips.

The tension system is a structure that is fabricated from a circular tensioning system that houses a mechanical locking system and the excess cable length. Two circular tensioning systems are built into the garment: one on the left hip and one on the right hip. Each system houses two cables. The cables are constructed from lightweight steel braids and coated with rubber. The main cable is approximately 3.5 feet in length. Attached to the main cable are a plurality of sub-cables, for example, five to seven (depending upon the size of the garment) sub-cables. The sub-cables attach to the main cable at approximately 90 degrees and extend to attach with the elastic bands. Located directly on the spine of the garment and directly between the left and right elastic bands is a solid plastic cam. There are cams located between each set of left and right elastic bands. The cam may be made of a hardened plastic and has 4 holes drilled though: up, down, left, and right. The holes that extend up and down are twice the size of the holes that extend left and right. The primary cables are fed through the up and down holes of the cam and extend to the upper thoracic vertebrae. The sub-cables are fed through the holes that extend left and right and allow the elastic bands to be tightened with the circular tensioning system.

The wearer can make coarse fit adjustments by manipulating the micro clips on the elastic bands themselves. After an initial adjustment, the wearer will don the garment and zip the zipper to the top. As lateral or bi-lateral adjustment is required or as the wearer feels greater or less tension is required, he or she will fine-tune the circular tensioning system to achieve the desired result. As the wearer tightens the system, the cable will be wound into the tensioning system. The main cable will be drawn downward and thus pull the sub-cables towards the center of the garment. This pulling action will create additional tension in the elastic bands. Furthermore, each side of the garment can be operated independently.

Referring now to the drawings, the garment is created with a locking zipper (1) in the middle of the garment that acts as centering point and a way to actually close the garment. The locking zipper is complemented by a rigid seam line/band attachment (2) that allows for bands to be attached via flatlock stitching (4). The bands increase in thickness from top to bottom (5), as this provides the greater torque required for straightening the lumbar region. Lastly, there are micro clips (6) or micro adjustment tabs that allow the band to be fed through the cable system and secured back to itself These same bands are sewn into the garment (on the inside) and are thus not visible but are being displayed prominently in the drawings for the sake of showing how the bilateral tension, mechanical tensioning system works.

Referring now to FIG. 2, the rigid seam line (1) extends all the way to the posterior of the garment as well. This seam line has band loops (2) attached to it that secure the band and allow the bands to feed through the cable system to be shorter or longer. There is also a cam for cable guidance and tension (3), the cable being part of the mechanical tension system that applies torque to the band. The tension wheel (4) located on the bottom of the garment is used to individually adjust the amount of tension across the bands. The cable system is fed through a channel (5) sewn into the garment that keeps it anchored and allows for variation of band length.

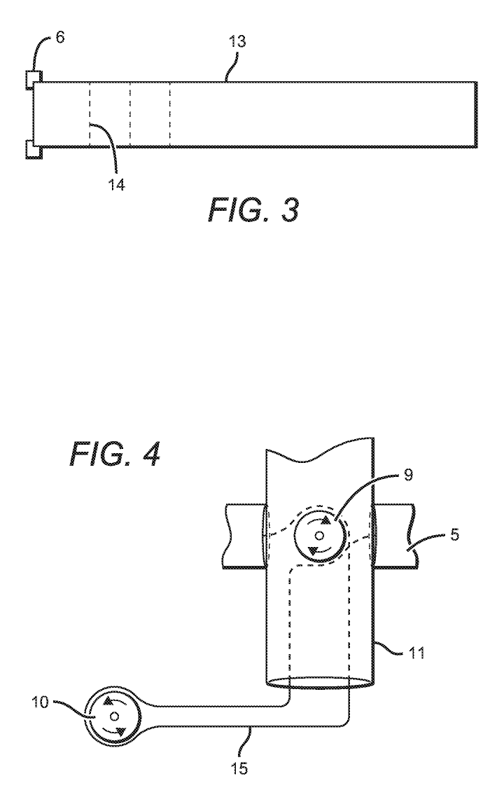

Referring now to FIG. 3, the individualized, sewn-into-the-garment bands enable the bilateral tension and intracavitary pressure in the garment. These bands contain:

a) Micro clips (1) that allow the band to be secured back to itself for adjustments;

b) The function of two-way stretch that allows for variable length, is micro-elastic, made from spandex and poly weave (2);

c) Clip point marks (3) that are used for interpolation.

Referring now to FIG. 4, a cable and tension (mechanical tensioning) system is shown that allows bands to be individually adjusted for different types of pressure and length (which also varies tension/pressure). The band (1) is attached to this system, which is fed through the garment channel (2), as described in FIG. 2. The tension wheel (3), cam (4), and cable (5) collectively allow wearers to turn the tension wheel to tighten or loosen the cable, and thus increase pressure on the band, to their desired level of tension in order to treat their specific spinal or thoracic deviations. The cam (4), with its four holes drilled through, holds the main cable and the sub-cables, with the main cables (5) running up and down and the sub-cables pulled through left and right and attached to the elastic bands (1) in order to create tension when pulled. This tension is created by turning the tension wheel (3), as described above, which wounds the cable into the tensioning system. The main cable will be drawn downward and thus pull the sub-cables towards the center of the garment. This pulling action will create additional tension in the elastic bands. Each side of the garment can be operated independently.

Having thus described several embodiments for practicing the inventive method, its advantages and objectives can be easily understood. Variations from the description above may and can be made by one skilled in the art without departing from the scope of the invention.

Accordingly, this invention is not to be limited by the embodiments as described, which are given by way of example only and not by way of limitation.

What is claimed is:

1. An adjustable garment, comprising:
a plurality of garment regions;
a mechanical tensioning system comprising a spool and a cable; and
at least one band comprising a first and a second end, said first end attached along a longitudinal center line of said garment and said second end attached to said mechanical tensioning system,
wherein said plurality of regions are individually adjustable using said mechanical tensioning system to provide a customized tension fit.

2. The garment of claim 1, wherein said at least one band extends through an internal loop of said garment.

3. The garment of claim 1, further comprising a plurality of bands.

4. The garment of claim 3, wherein said plurality of bands are arranged horizontally across said garment.

5. The garment of claim 3, wherein said plurality of garment regions comprise a lumbar region and a thoracic region.

6. The garment of claim 5, wherein said plurality of bands are stacked in a parallel arrangement from said lumbar region to said thoracic region.

7. The garment of claim 1, further comprising a micro adjustment tab on said second end of said at least one band.

8. The garment of claim 7, wherein said micro adjustment tab comprises a hook-and-loop fastening mechanism.

9. The garment of claim 7, wherein said micro adjustment tab comprises a plurality of micro clips.

10. The garment of claim 1, wherein said garment is a vest.

11. The garment of claim 1, wherein said garment is a waistband.

12. The garment of claim 1, wherein said garment is a girdle.

13. A circular tensioning system, comprising:
a tension wheel;
a main cable wound around said tension wheel;
a plurality of sub-cables attached to said main cable;
at least two bands each attached to at least one of said plurality of sub-cables; and
a cam positioned between said at least two bands comprising a plurality of holes, said main cable extending through a first and second of said holes and said sub-cables extending through a third and fourth of said holes,
wherein said at least two bands are adjustable by turning said tension wheel.

14. The tensioning system of claim 13, wherein said main cable comprises a steel braid.

15. The tensioning system of claim 13, wherein said main cable is rubber coated.

16. The tensioning system of claim 13, wherein said sub-cables are orthogonal to said main cable.

17. A method for using an adjustable garment, comprising:
adjusting a plurality of regions of said garment by adjusting a length of at least one band within at least one of said regions;
securing said garment to the body of an individual; and
adjusting the tension applied to at least one of said plurality of regions by a mechanical tensioning system to provide a customized tension fit.

18. The method of claim 7, further comprising adjusting a first side of said garment independently from a second side of said garment.

19. The method of claim 17, further comprising feeding said at least one band through said mechanical tensioning system and securing said at least one band back to itself with a micro adjustment tab on an end of said at least one band.

20. The method of claim 17, wherein adjusting the length of said at least one band occurs prior to securing said garment to the body of said individual.
